(19) United States
(12) Patent Application Publication
(10) Pub. No.: US 2003/0121768 A1
(43) Pub. Date: Jul. 3, 2003

(54) Alternate extraction solvent for acrylic acid purification process

(76) Inventors: Salvador Aldrett Lee, Corpus Christi, TX (US); Diane Elizabeth Allen, Corpus Christi, TX (US); Christopher Lawrence Wilkins, Mathis, TX (US)

Correspondence Address: M. Susan Spiering, c/o Celanese Ltd., P.O. Box 9077, Corpus Christi, TX, 78409, US

(21) Appl. No.: 10/021,927

(22) Filed: Dec. 13, 2001

(51) Int. Cl. B01D 3/36; C07C 51/44

(52) U.S. Cl. 203/43; 203/46; 562/600

(57) ABSTRACT

A process for the separation and purification of acrylic acid is disclosed, wherein ethyl propionate is employed as an extraction solvent.

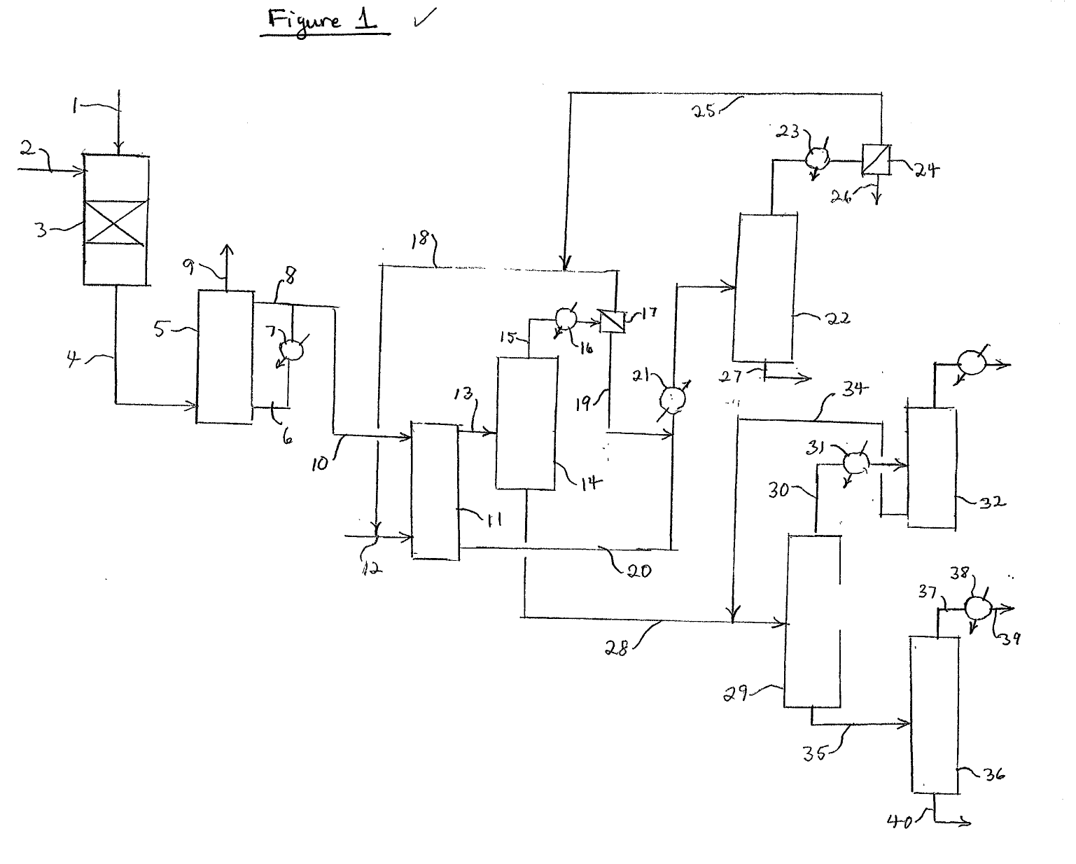

### BACKGROUND OF THE INVENTION

1. Field of the Invention

This invention relates to an improved acrylic acid purification process including solvent extraction and azeotropic distillation.

2. Description of the Related Art

Acrylic acid is an important compound useful as an intermediate in the production of a wide variety of acrylic resins. Acrylic acid can be used as is to produce polymers for different uses or it can be further reacted to produce derivatives such as esters and salts which can serve as monomers suitable for polymerization. Acrylic acid or its derivatives can be polymerized by themselves to form homopolymers or with other monomers to form copolymers. These polymers can be molded or cast to obtain many different forms of useful solid objects or they can be employed to obtain useful liquid compositions such as water-based or organic solvent-based paints, varnishes or textile treating compositions.

It is known in the art to produce acrylic acid by catalytic oxidation of propylene and/or acrolein with oxygen in the vapor phase. In a two stage process, for example, propylene is oxidized primarily to acrolein, and then the reaction of acrolein to acrylic acid occurs in a second stage, as shown in the following equations:

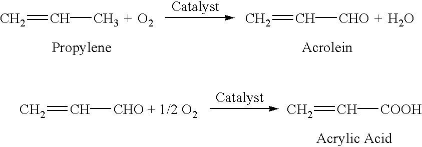

Alternatively, propylene or acrolein may be catalytically oxidized in a single vapor phase stage to obtain acrylic acid.

Because the foregoing oxidation reactions are highly exothermic, the product of the process is a hot gaseous effluent comprising acrylic acid and various other components. Various sources can be employed for the sources of oxygen. For example, air, pure oxygen, or blend gases. Air includes fairly large percentages of nitrogen, and oxygen and smaller percentages of other components such as carbon dioxide and argon. The by-products of the acrylic acid reaction may vary based on the source of oxygen, or the raw materials fed to the reaction. Other by-products to contend with in the acrylic acid reaction involve use of propylene which also contains impurity(ies) in commercial supplies. The gaseous effluent of the acrylic acid reaction must be treated to obtain a highly pure acrylic acid. Increased purity is desired due to the use of acrylic acid in the production of such products as acrylate esters and acrylic resins. Those of skill in the art will recognize method to treat the impurities of the acrylic acid or the feed materials therefor.

The separation and purification of acrylic acid from the hot gaseous effluent produced by the oxidation reaction generally requires the utilization of a combination of unit operations such as condensation, evaporation, distillation, solvent extraction, heat and mass transfer, decantation, etc. An additional consideration in the separation and purification process of acrylic acid is the use of extraction solvents and their suitability in the environment.

U.S. Pat. No. 4,554,054 issued Nov. 19, 1985 to R. W. Coyle, discloses a process to recover acrylic acid or methacrylic acid by using a split quench process wherein the gaseous effluent is condensed in a first stage condenser to form a relatively concentrated aqueous acrylic or methacrylic acid liquid stream, and the vapor phase leaving the first stage condenser is condensed in a second stage condenser to form an aqueous acrylic or methacrylic acid liquid stream less concentrated than the first stage condenser liquid stream, with the liquid streams from both condensers being fed to separate first and second distillation columns respectively to remove acrolein or methacrolein as overhead products. The liquid stream from the bottom of the second distillation column is subjected to solvent extraction to separate acrylic or methacrylic acid, wherein the solvent employed can be ethyl propionate.

Japanese Patent 45026485, issued Sep. 1, 1970, discloses the separation of acrylic acid from an aqueous solution obtained from the oxidation in the gaseous phase of propylene and acrolein, by solvent extraction using ethyl propionate as extractant. The extractant can be dehydrated azeotropically.

### BRIEF SUMMARY OF THE INVENTION

In accordance with this invention, acrylic acid is separated and purified from a gaseous effluent stream produced by catalytic oxidation of propylene and/or acrolein, said gaseous effluent comprising acrylic acid and water and some unreacted acrolein. The process comprises contacting:

a) propylene and/or acrolein with oxygen in the presence of an oxidation catalyst to form a gaseous effluent or stream of acrylic acid, water, unreacted acrolein and by-products;

b) contacting the gaseous stream in a quenching zone with liquid previously formed by condensation in such quenching zone to form an aqueous acrylic acid stream;

c) directing the acrylic acid containing stream from the quenching zone of step (b) to a solvent extraction zone where it is contacted with ethyl propionate as the extractant to form an extract;

d) forwarding the extract comprising primarily of acrylic acid and ethyl propionate solvent with some acetic acid and water to an azeotropic distillation zone where the extract is distilled to obtain an ethyl propionate/water azeotrope as overhead and a crude acrylic acid as a residue stream; and,

e) further purifying the crude acrylic acid.

This invention employs ethyl propionate (EP) as extractant which allows for the more efficient operation of the unit. EP is less soluble in water than a solvent such as ethyl acrylate, and when separated some of the ethyl acrylate generally goes into the water phase causing a need to remove the ethyl acrylate from the process. Ethyl acrylate has a tendency to polymerize in the process and so requires additional processing and operation to keep the acrylic acid production/purification process flowing efficiently.

### BRIEF DESCRIPTION OF THE DRAWING

The drawing is a schematic diagram of the process of the invention for separating and purifying acrylic acid from the gaseous effluent produced by the two stage catalytic oxidation of propylene with the oxygen in air.

### DETAILED DESCRIPTION OF THE INVENTION

The gaseous effluent from which acrylic acid is separated and purified by the process of the invention is preferably produced from a two stage catalytic oxidation process in the vapor phase, employing oxygen and a catalyst and wherein propylene is oxidized in the first stage primarily to acrolein, and most of the acrolein is oxidized to acrylic acid in the second stage.

In such a two stage oxidation process, acrylic acid is generally present in the gaseous effluent in an amount, for example of about 10 to about 20 wt %, preferably about 14 to about 16 wt %, and acrolein may be present as a portion of the product of the first stage oxidation remaining unreacted in the second stage. The amount of such acrolein present is preferably no more than about 1.0 wt % based on the weight of the acrylic acid present in the oxidation effluent and preferably as close as possible to zero.

Although the reaction herein is described in a one or two stage reaction process, it is understood that any reaction type will work. The present invention is directed to the use of ethyl propionate as extractant, and the separation and purification of acrylic acid.

When employing air as the oxygen source, the gaseous oxidation effluent also contains substantial amounts of noncondensable nitrogen, e.g. about 45 to about 55 wt % originating in the air intake to the oxidation reaction, and water, e.g. about 25 to about 30 wt %, originating from the steam used in the reaction process as diluent. Also present in the oxidation effluent may be small amounts of by-products of the oxidation reactions such as acetic acid, aldehydes and other organic acids. Also present may be small amounts, a total of no more than about 8 wt % based on the total weight of the gaseous effluent, of noncondensable gases other than nitrogen. The noncondensable gases include unreacted oxygen, part of the carbon dioxide and argon, which originate in the air intake. Other by products of the reaction or originating in the propylene intake, are unreacted propylene, propane, carbon dioxide, and carbon monoxide. The by-products of the acrylic acid reaction may vary based on the source of oxygen employed, the process parameters employed, etc.

More specific by-products produced include aldehydes such as furfural, benzaldehyde, acrolein, and acids such as acetic acid and propionic acid.

All of the weight percents given previously are based on the total weight of the gaseous oxidation effluent.

In another embodiment of the invention, the separation and purification process of the invention may also be applied to the acrylic acid containing gaseous effluent from a single stage oxidation of an acrolein rich feed in which case the gaseous effluent composition may be somewhat different than that obtained from the two stage oxidation of propylene. The separation and purification process of this invention is operable for the treatment of gaseous effluents from both types of oxidation processes.

EP can be used or be applied to any acrylic acid process where one has an excess of water present. “Excess” means about or greater than 30 wt % of water based on the total composition of the quench residue stream.

The operations specified in the following description of the process of this invention are generally carried out at reduced pressure. However, it is within the skill of the art to utilize atmospheric or superatmospheric pressure when necessary to accomplish a particularly desired result.

The initial process of the invention, i.e., the reaction of propylene and/or acrolein to acrylic acid may be carried out by a variety of methods as described herein. As mentioned previously, the invention is directed to the use of EP solvent as extractant in the reaction process. The acrylic acid produced by whatever process must be separated and purified. The art has typically employed solvents such as ethyl acrylate, benzene, toluene, and the like, for such extractants. EP has been found to be easier to work with, does not have the polymerization concerns of ethyl acrylate or the odor associated with ethyl acrylate. EP is considered environmentally friendly due in part to its high odor threshold and non-carcinogenic character. EP is as good or better to use as ethyl acrylate from a cost perspective and does not have the regulatory requirements currently associated with toluene or benzene.

When employing propylene and/or acrolein with air, the resulting stream of gaseous oxidation effluent will be directed to a quenching tower. The resulting quenching of the oxidation effluent causes the condensation of the acrylic acid and water in the effluent with the noncondensable gases, acrolein and most of any by-product aldehydes and some of the by-product other organic acids being withdrawn by the overhead vent system. Polymerization inhibitor is usually added to the quench tower.

That portion of the cooled condensate from the quench tower which is not fed back into the tower as quenching liquid, is directed to an extraction zone, e.g. a staged extraction tower where it is contacted, typically, countercurrently with ethyl propionate as extraction solvent in the extraction tower. Generally, EP can be employed as a solvent: feed ratio of about 0.70-1.00, or preferably about 0.75-0.85 ratio. The actual amount employed will vary based on water present in the system as the stream enters the extraction zone. The cooled condensate from the quench tower fed to the extraction tower is composed primarily of acrylic acid and water and contains, e.g., about 30 to about 40 wt % of acrylic acid and about 2 to about 4 wt % of acetic acid with most of the remainder as water. The extraction is carried out at a temperature, e.g., of about 20 to about 30° C. Such extraction accomplishes the transfer of organic acids including acrylic and acetic acids, from the aqueous phase into the organic extract phase which is withdrawn from the extraction tower and comprises about 25 to about 30 wt % acrylic acid, about 10 to about 15 wt % of water, about 50 to about 60 wt % ethyl propionate solvent, and about 1 to about 5 wt % acetic acid. Such organic phase extract is fed to an azeotropic distillation zone, e.g., a crude product fractionation tower. Preferably, the aqueous raffinate containing about 1 to about 3 wt % of extraction solvent with most of the remainder water, is fed to a solvent recovery zone, e.g., a solvent recovery fractionation tower.

The azeotropic distillation zone or crude product fractionation tower, is where crude acrylic acid is separated from extraction solvent in the organic extract phase taken from the extraction tower. A vaporized azeotrope of water and ethyl propionate extraction solvent leaves the crude product tower, and is further treated to effect re-use of the solvent. Preferably the vaporized azeotrope is condensed, and the condensate separated into an organic phase comprising, primarily ethyl propionate extraction solvent which is recycled to the extraction tower, and an aqueous phase comprising primarily water and a small amount of extraction solvent. The latter stream may be combined with the aqueous raffinate from the extraction tower having a similar composition and the combined stream fed to the solvent recovery tower.

A stream from the base of the crude product tower is crude acrylic acid containing about 90 to about 96 wt % of acrylic acid and about 3 to about 8 wt % of acetic acid, and may be treated further to obtain acrylic acid products of higher purity. Polymerization inhibitors are usually added to the crude product tower.

It is preferred to feed the aqueous raffinate from the extraction tower and the aqueous phase of the overhead condensate from the crude product tower to the solvent recovery tower where most of the ethyl propionate extraction solvent is separated from the water in these streams. The combined stream entering the solvent recovery tower is primarily water containing small amounts of extraction solvent, e.g., about 1 to about 5 wt % of the extraction solvent. The tower is operated so that a stream comprising mainly water and small amounts of organic compounds is withdrawn and sent to waste water treatment. The overhead vapors comprising water and about 17 to about 22 wt % of ethyl propionate extraction solvent, is condensed and separated into an organic and aqueous phase. The organic phase comprises primarily extraction solvent and is recycled to the extraction tower, and the aqueous phase which is relatively pure water may be sent to steam generation or otherwise disposed of.

The crude acrylic acid from the base of the crude product tower is generally treated to raise the purity of the acrylic acid. For example, the acrylic acid may be treated in a light ends separation zone, e.g., a light ends fractionation tower. Thus, the crude acrylic acid may be fed to such tower which may be operated so that the temperature at the base is about 105° C. to about 110° C. and the overhead temperature is about 75° C. to about 85° C. The residue withdrawn from the base of this tower is acrylic acid of higher purity than the acrylic acid fed to the light ends tower. The acrylic acid residue contains about 95.6 to about 99.8 wt % of acrylic acid, while the overhead condensate is a mixture containing about 20 to about 80 wt % of acrylic acid, about 8 to about 20 wt % of acetic acid, and a trace amount of water. In some systems, this overhead stream may be recycled to the crude product fractionation tower from which the acetic acid is withdrawn with the extraction solvent/water azeotrope and results in the waste water residue, and the decanted aqueous phase of the overhead of the solvent recovery tower. Preferably, however, the overhead condensate from the light ends tower is fed to an acetic acid recovery zone, e.g., an acetic acid fractionation tower which is operated at a bottom temperature of, e.g., about 90° C. to about 95° C., and an overhead temperature of, e.g., about 60° C. to about 65° C. Light distillate materials from this tower, primarily acetic acid, are removed as overhead, while the residue acrylic acid is recycled as feed to the light ends tower. Polymerization inhibitors are usually added to the light ends and acetic acid recovery tower.

It is well known to those of skill in the art that temperature parameters vary from the top to the bottom of given towers. The exact temperature can be determined by methods well known in the art, and further will vary based on the exact composition of the materials fed, and the operation parameters such as pressure.

To produce higher purity (e.g.,>99.8%) glacial acrylic acid, the acrylic acid residue from the light ends tower may be fed to a glacial acrylic acid finishing zone, e.g., a finishing fractionation tower. The overhead condensate is a highly pure acrylic acid containing, e.g., at least about 99.5 wt % of acrylic acid which is suitable as the feed to a crystallizer purification train for production of glacial acrylic acid, while the residue is an ester grade acrylic containing, e.g., at least 75 wt % acrylic acid suitable as feed to an ester production unit.

Turning now to the drawing which shows a preferred embodiment of the process of this invention, propylene and air are fed through lines 1 and 2 to catalytic reactor 3 in which the oxidation of propylene to acrylic acid with the oxygen in air is carried out in two catalytic oxidation stages, resulting in the formation of a gaseous effluent containing at least about 10 wt % of acrylic acid and no more than about 1 wt % of acrolein based on the weight of acrylic acid. Such gas is fed through line 4 to quench tower 5 at the base of the tower. Condensed hot liquid is pumped from the base of tower 5 through line 6 and cooler 7 with most of the resulting cooled liquid flowing through line 8 back to tower 5 where it contacts rising hot gas from reactor 1 and acts to condense the higher boiling components of the gas including acrylic acid and water, which flow to the base of tower 5. The noncondensable and lower boiling components of the gas are vented through line 9.

Part of the liquid from cooler 7 is fed through line 10 to a point near the top of extraction tower 11 where it contacts countercurrently ethyl propionate extraction solvent entering tower 11 through line 12 near the base of the tower. This facilitates the extraction by the solvent of most of the organics including acrylic acid and some acetic acid in the condensed reactor effluent. The resulting extract leaves tower 11 through line 13 and is fed to crude product tower 14 which separates the extract into a low boiling water/solvent azeotrope, vapors of which are withdrawn from the top of tower 14 through line 15. These vapors are condensed in cooler 16 and are fed to decanter 17 in which the condensate separates into two phases, an organic phase comprising primarily extraction solvent which is recycled through line 18 to solvent input line 12 leading to extraction tower 11, and an aqueous phase comprising primarily water and a small amount of extraction solvent withdrawn from decanter 17 through line 19. The latter stream is combined with the aqueous raffinate also comprising primarily water and a small amount of extraction solvent, which leaves the base of extraction tower 11 through line 20, and the combined streams are fed through solvent recovery tower feed preheater 21 to solvent recovery tower 22. The overhead vapors from tower 22 are composed primarily of a solvent/water azeotrope and are condensed in cooler 23 and separated in decanter 24 into an organic phase which is primarily extraction solvent withdrawn through line 25 and combined with the organic phase in line 18 from decanter 17 of crude product tower 14 and the solvent input to extraction tower 11 in line 12. The aqueous phase from decanter 24 is primarily water which is withdrawn through line 26 and may be sent to the steam generator or otherwise disposed of. The residue from solvent recovery tower 22 is primarily water containing small amounts of organics which is withdrawn through line 27 and sent to waste water treatment.

The residue from crude product tower 14 is crude acrylic acid withdrawn through line 28 to light ends tower 29 which separates most of the acetic acid impurity from the crude acrylic acid. Overhead vapors from tower 29 comprising primarily acetic and acrylic acids are withdrawn through line 30, are condensed in cooler 31 and the condensate fed to acetic acid recovery tower 32. The overhead vapors from tower 32 are primarily acetic acid and are condensed in cooler 33 and sent to further treatment or disposed of. The residue from tower 32 comprises primarily acrylic acid and is recycled through line 34 to light ends tower 29.

For the production of glacial acrylic acid, the somewhat less pure acid obtained as a residue in light ends tower 29 is fed through line 35 to finishing tower 36 from which overhead vapors are withdrawn through line 37 and condensed in cooler 38 to obtain a highly pure acrylic acid which is forwarded through line 39 to a crystallizer train for the production of the glacial acid. The residue is ester grade acrylic acid withdrawn from the tower through line 40.

The example and the data herein is directed to the utility and suitability of the ethyl propionate in an acrylic acid purification system.

Various data were developed to show the suitability of ethyl propionate utilized in the process of this invention as the extraction solvent. For example, one group of data resulted from the determination of liquid-liquid equilibrium (LLE) values for 34% aqueous acrylic acid (HAcA) in a known amount of solvent. These LLE experiments were conducted in triplicate by the following procedure: 50 grams of a 34% by weight aqueous acrylic acid solution and 50 grams of solvent were charged to a separatory funnel. The mixture was shaken for 3 minutes at room temperature and the aqueous and organic phases were allowed to separate. Each layer was weighed and analyzed by gas chromatography for acrylic acid and solvent content. The amount of water was determined by Karl Fischer titration or by difference.

Results of the LLE experiments are shown in Table I. The distribution coefficients were calculated as the wt % acrylic acid in the organic layer divided by the wt % acrylic acid in the aqueous layer. Selectivity was defined as the wt % acrylic acid in the organic layer divided by the wt % water in the organic layer. These definitions can be expressed as follows:

$$\begin{matrix}Distribution Coefficient = \frac{wt \% HAcA in organic layer}{wt \% HAcA in aqueous layer} \\ \\ Selectivity = \frac{wt \% HAcA in organic layer}{wt \% water in organic layer}\end{matrix}$$

TABLE I
Ethyl Propionate as the Solvent

| Sample | Type of | Analytical Results | | | Distribution | |
|---|---|---|---|---|---|---|
| No. | Sample | % Water | % Acrylic Acid | % Ethyl Propionate | Coeff. | Selectivity |
| 1 | Aqueous | 89.81 | 9.17 | 1.02 | 2.9662 | 3.4783 |
| 2 | Organic | 7.82 | 27.20 | 64.98 | | |
| 3 | Aqueous | 89.32 | 9.62 | 1.06 | 2.8794 | 3.3905 |
| 3 | Organic | 8.17 | 27.70 | 64.13 | | |
| 3 | Aqueous | 89.29 | 9.70 | 1.01 | 2.8763 | 3.4024 |
| 6 | Organic | 8.20 | 27.90 | 63.90 | | |
| | | | | Average | 2.9133 | 3.4237 |

TABLE II
Ethyl Acrylate as Solvent

| | | | |
|---|---|---|---|
| Sample | Type of | Analytical Results | Distribution |

| | | | | | | |
|---|---|---|---|---|---|---|
| No. | Sample | % Water | % Acrylic Acid | % Ethyl Acrylate | Coeff. | Selectivity |

| | | | | | | |
|---|---|---|---|---|---|---|
| 1 | Aqueous | 88.95 | 8.78 | 2.27 | 2.8018 | 3.0483 |
| 2 | Organic | 8.07 | 24.60 | 67.33 | | |
| 3 | Aqueous | 88.78 | 8.98 | 2.24 | 2.7840 | 3.0414 |
| 4 | Organic | 8.22 | 25.00 | 66.78 | | |
| 5 | Aqueous | 88.49 | 9.20 | 2.31 | 2.5217 | 2.8431 |
| 6 | Organic | 8.16 | 23.20 | 68.64 | | |
| | | | | Average | 2.7025 | 2.9776 |

As can be seen from the data in Tables I and II, ethyl propionate compares favorably to ethyl acrylate as an extraction solvent for acrylic acid. Ethyl propionate has a higher distribution coefficient and higher selectivity for acrylic acid than does ethyl acrylate, resulting in higher yields of acrylic acid in the extraction tower organic extract phase and lower solvent losses to the aqueous phase.

Using the procedure previously described, additional LLE data over a range of compositions were determined for ethyl propionate as extraction solvent. As before, the experiments were conducted in triplicate. In all the experiments, 40 grams of solvent and 60 grams of water were employed but the amount of acrylic acid was 10 grams for the first group of three experiments, 35 grams for the second group, 50 grams for the third group, and 60 grams for the fourth group. The temperature was approximately room temperature for the experiments illustrated. Analytical data for the three components (i.e. % water, % acrylic acid, and % ethyl propionate) in both the aqueous and organic phases were determined, and the distribution coefficient and selectivity were calculated for each experiment. The results are shown in Table III.

TABLE III
Ethyl Propionate as the Solvent

| | | wt % of | Analytical Results | | | | |
|---|---|---|---|---|---|---|---|
| Sample | Type of | Acrylic | | % Acrylic | % Ethyl | Distribution | |
| Number | Sample | Acid (g) | % Water | Acid | Propionate | Coeff. | Selectivity |
| 1 | Aqueous | 10.0 | 91.90 | 6.45 | 1.70 | 2.0930 | 1.9014 |
| 2 | Organic | 10.0 | 7.10 | 13.50 | 79.40 | | |
| 3 | Aqueous | 10.0 | 91.50 | 6.70 | 1.76 | 2.0896 | 2.1875 |
| 4 | Organic | 10.0 | 6.40 | 14.00 | 79.60 | | |
| 5 | Aqueous | 10.0 | 91.70 | 6.60 | 1.70 | 2.0606 | 2.1587 |
| 6 | Organic | 10.0 | 6.30 | 13.60 | 80.10 | | |
| 7 | Aqueous | 35.0 | 79.00 | 18.60 | 2.44 | 1.6935 | 1.3235 |
| 8 | Organic | 35.0 | 23.80 | 31.50 | 44.70 | | |
| 9 | Aqueous | 35.0 | 79.30 | 18.30 | 2.40 | 1.9508 | 2.4965 |
| 10 | Organic | 35.0 | 14.30 | 35.70 | 50.00 | | |
| 11 | Aqueous | 35.0 | 79.00 | 18.60 | 2.44 | 1.9086 | 2.3986 |
| 12 | Organic | 35.0 | 14.80 | 35.50 | 49.70 | | |
| 13 | Aqueous | 50.0 | 75.80 | 22.20 | 2.04 | 1.7748 | 1.5096 |
| 14 | Organic | 50.0 | 26.10 | 39.40 | 34.50 | | |
| 15 | Aqueous | 50.0 | 71.30 | 25.60 | 3.06 | 1.5469 | 1.5290 |
| 16 | Organic | 50.0 | 25.90 | 39.60 | 33.70 | | |
| 17 | Aqueous | 50.0 | 71.10 | 25.80 | 3.08 | 1.5271 | 1.4593 |
| 18 | Organic | 50.0 | 27.00 | 39.40 | 33.60 | | |
| 19 | Aqueous | 60.0 | 63.00 | 31.50 | 5.48 | 1.2946 | 1.2137 |
| 20 | Organic | 60.0 | 33.60 | 40.78 | 25.64 | | |
| 21 | Aqueous | 60.0 | 63.40 | 31.30 | 5.28 | 1.3000 | 1.2293 |
| 22 | Organic | 60.0 | 33.10 | 40.69 | 26.18 | | |
| 23 | Aqueous | 60.0 | 63.20 | 31.30 | 5.46 | 1.2895 | 1.1733 |
| 24 | Organic | 60.0 | 34.40 | 40.36 | 25.22 | | |

The results shown in Table I to III, particularly the values of distribution coefficient and selectivity, indicate that ethyl propionate is a satisfactory to superior solvent for the extraction of acrylic acid from the feed to the separation and purification process of this invention.

We claim:

1. A process for the separation of acrylic acid in the gaseous effluent stream produced by the catalytic oxidation of propylene and/or acrolein said gaseous effluent, comprising acrylic acid water and some unreacted acrolein, the process comprises contacting:
a) propylene and/or acrolein with oxygen in the presence of an oxidation catalyst to form a gaseous effluent or stream of acrylic acid, water, unreacted acrolein and by-products;
b) contacting the gaseous stream in a quenching zone with liquid previously formed by condensation in such quenching zone to form an aqueous acrylic acid stream;
c) directing the acrylic acid containing stream from the quenching zone of step
b) to a solvent extraction zone where it is contacted with ethyl propionate as the extractant to form an extract, and
d) forwarding the extract comprising primarily of acrylic acid and ethyl propionate solvent with some acetic acid and water to an azeotropic distillation zone where the extract is distilled to obtain an ethyl propionate/water azeotrope as overhead and a crude acrylic acid as a residue stream.

2. The process of claim 1 wherein the gaseous effluent is produced from a two-stage catalytic oxidation process in the vapor phase.

3. The process of claim 1 wherein acrylic acid is present in said gaseous effluent stream in an amount of about 10 to about 20 wt % based on the total weight of the stream.

4. The process of claim 2 wherein acrolein is present as a portion of the product of the first stage oxidation in an amount no greater than about 1 wt % based on the weight of the acrylic acid present in the oxidation effluent.

5. The process of claim 1 wherein the gaseous effluent is produced from a single stage oxidation process.

6. The process of claim 1 wherein the acrylic acid containing stream of step (c) is contacted countercurrently with the ethyl propionate extraction solvent.

7. The process of claim 6 wherein the extraction is conducted at a temperature of about 20° C. to about 30° C.

8. The process of claim 1 wherein the distillation of the extract of step (d) comprises condensing a vaporized azeotrope of water and ethyl propionate extraction solvent, and separating into an organic and aqueous phase.

9. The process of claim 8 wherein the organic phase is directed to an extraction tower.

10. The process of claim 1 wherein the crude acrylic acid of step (d) is directed to a light ends tower separation zone.

11. The process of claim 1 wherein the solvent: feed ratio of EP as extractant is about 0.70-1.00.

12. The process of claim 11 wherein the ratio of EP as extractant is about 0.75-0.85.
